(19) United States
(12) Patent Application Publication
(10) Pub. No.: US 2026/0041874 A1
(43) Pub. Date: Feb. 12, 2026

(54) AROUSAL STATE CONTROL SYSTEM, WEARABLE DEVICE, AND AROUSAL STATE CONTROL METHOD

(71) Applicants: Koji IWASAKI, Kanagawa (JP); Yoshihiro SEKINE, Aichi (JP); Ippei KIMURA, Osaka (JP); Ryohju TOGAMI, Osaka (JP); Masato YOSHIKAWA, Tokyo (JP)

(72) Inventors: Koji IWASAKI, Kanagawa (JP); Yoshihiro SEKINE, Aichi (JP); Ippei KIMURA, Osaka (JP); Ryohju TOGAMI, Osaka (JP); Masato YOSHIKAWA, Tokyo (JP)

(21) Appl. No.: 19/126,263

(22) PCT Filed: Nov. 16, 2023
(86) PCT No.: PCT/IB2023/061584

§ 371 Date: May 1, 2025

(30) Foreign Application Priority Data
Nov. 30, 2022 (JP) 2022-192273
Oct. 5, 2023 (JP) 2023-173627

(51) Int. Cl. A61M 21/00; A61F 7/00; A61N 1/36

(52) U.S. Cl. CPC A61M 21/00; A61F 7/00; A61N 1/36031; A61F 2007/0011; A61F 2007/0093; A61M 2021/0022; A61M 2021/0066; A61M 2021/0072; A61M 2021/0083; A61M 2205/3303

(57) ABSTRACT

An arousal state control system includes a sensor, a stimulation control unit, and a stimulator. The sensor acquires biological information of a user. The stimulation control unit determines, based on the biological information, one or more types of stimuli to control a state of arousal of the user. The stimulator applies the one or more types of stimuli determined by the stimulation control unit to the user.

### TECHNICAL FIELD

Embodiments of the present disclosure relate to an arousal state control system, a wearable device, and an arousal state control method.

### BACKGROUND ART

The autonomic nervous system is a nervous system that regulates autonomic functions such as respiration, digestion, circulatory, and body temperature maintenance. The balance between sympathetic nerves and parasympathetic nerves maintains the autonomic nervous system. Sympathetic nerves become active during arousal, whereas parasympathetic nerves become active during relaxation. Although high levels of arousal increase concentration, an excessively high level of arousal reduces performance due to excessive stress. Such a relationship between arousal and performance is known as the Yerkes-Dotson law.

A stress suppression system has been proposed that measures biological information such as the body temperature and the heart rate of a user to induce a healing effect by, for example, images, music, or tactile stimulation (see, for example, Patent Literature (PTL) 1).

### CITATION LIST

### Patent Literature

[PTL 1]
Japanese Unexamined Patent Application Publication No. 2020-202971

### SUMMARY OF INVENTION

### Technical Problem

According to the Yerkes-Dotson law, the level of arousal and performance exhibits an inverted U-shaped correlation in a graph with the horizontal axis representing the level of arousal and the vertical axis representing the performance. The graph has a range of levels of arousal suitable for maximizing performance, whereas the performance decreases outside the range. This inverted U-shaped correlation varies depending on the difficulty of the work or tasks being addressed. For simple tasks, a certain degree of tension increases the productivity. For complex tasks, relaxing tension may improve efficiency. The appropriate level of arousal varies between individuals. To enhance intellectual productivity, a technique for controlling the state of arousal as appropriate for each user is desired in addition to the healing effect.

It is an object of the present invention to provide a technique for controlling the state of arousal as appropriate for each situation.

### Solution to Problem

An arousal state control system includes a sensor, a stimulation control unit, and a stimulator. The sensor acquires biological information of a user. The stimulation control unit determines, based on the biological information, one or more types of stimuli to control a state of arousal of the user. The stimulator applies the one or more types of stimuli determined by the stimulation control unit to the user.

A wearable device includes a body, a first stimulation unit, and a second stimulation unit. The first stimulation unit is disposed at a first portion of the body to apply a first stimulus. The second stimulation unit is disposed at a second portion of the body to apply a second stimulus different from the first stimulus. One or both of the first stimulation unit and the second stimulation unit are driven based on biological information of a user wearing the body. An arousal state control method includes acquiring biological information of a user, analyzing the biological information with an information processing device, determining, with the information processing device, one or more types of stimuli based on the analyzed biological information, to control a state of arousal of the user, and applying the determined one or more types of stimuli to the user.

### Advantageous Effects of Invention

The state of arousal of users can be controlled as appropriate.

### BRIEF DESCRIPTION OF DRAWINGS

A more complete appreciation of embodiments of the present disclosure and many of the attendant advantages and features thereof can be readily obtained and understood from the following detailed description with reference to the accompanying drawings.

FIG. 1 is a schematic diagram of an arousal state control system according to an embodiment of the present disclosure.

FIG. 2 is a schematic diagram illustrating a wearable device worn by a user, according to an embodiment of the present disclosure.

FIG. 3A is a diagram of a wearable device according to a first example of the present disclosure.

FIG. 3B is a diagram of a wearable device according to a second example of the present disclosure.

FIG. 3C is a diagram of a wearable device according to a third example of the present disclosure.

FIG. 4 is a block diagram illustrating a hardware configuration of an information processing device according to an embodiment of the present disclosure.

FIG. 5 is a functional block diagram of a stimulation control unit according to an embodiment of the present disclosure.

FIG. 6A is a flowchart of control executed by a stimulation control unit according to an embodiment of the present disclosure.

FIG. 6B is a continuation of the flowchart of FIG. 6A.

FIG. 7 is a table illustrating the result of evaluations of arousal state control.

FIG. 8 is a diagram of a wearable device according to another example of the present disclosure.

FIG. 9 is a diagram illustrating a configuration of a skin-contact side of the wearable device of FIG. 8.

FIG. 10 is a diagram of a wearable device according to yet another example of the present disclosure.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DESCRIPTION OF EMBODIMENTS

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

For the sake of simplicity, like reference signs denote like elements such as parts and materials having the same functions, and redundant descriptions thereof are omitted unless otherwise required. The sizes and relative positions of the components illustrated in the drawings may be exaggerated to facilitate understanding of the invention. As used herein, the term “connected/coupled” includes both direct connections and connections in which there are one or more intermediate connecting elements.

In the embodiments of the present disclosure, appropriate tension is given to a user, in addition to a relaxing effect, based on biological information to lead the user to an appropriate state of arousal. To achieve this, the biological information of the user is analyzed to generate and apply different types of stimuli. For example, a first stimulus for increasing tension or arousal is combined with a second stimulus for reducing tension or arousal. The state of arousal of the user is controlled as appropriate by simultaneous or separate application of different types of stimuli, to enhance intellectual productivity.

A description is given of configurations of an arousal state control system and a wearable device according to an embodiment of the present disclosure.

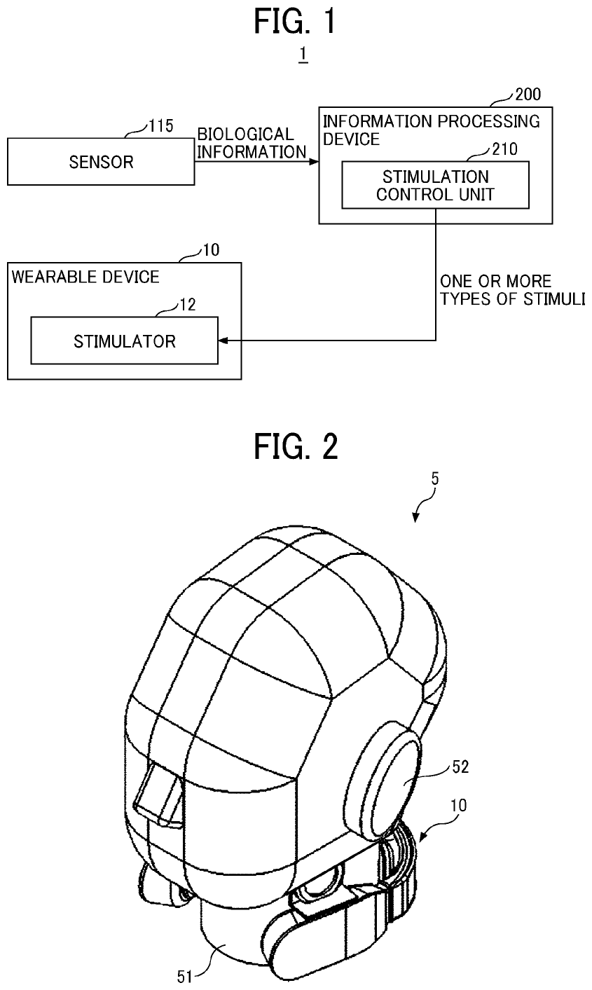

FIG. 1 is a schematic diagram of an arousal state control system 1 according to the present embodiment. An arousal state control system 1 includes a sensor 115, a stimulation control unit 210, and a stimulator 12. The sensor 115 acquires biological information of a user. The stimulation control unit 210 determines one or more types of stimuli to control the state of arousal based on the biological information. The stimulator 12 applies the one or more types of stimuli determined by the stimulation control unit 210 to the user. The stimulator 12 is implemented in the form of a wearable device 10. When the user wears the wearable device 10 and performs work, the user can be stimulated as appropriate for arousal during work.

The wearable device 10, the sensor 115, and an information processing device 200 may be separate devices. Alternatively, at least one of the sensor 115 and the stimulation control unit 210 may be incorporated in the wearable device 10. The information processing device 200 may be connected to the sensor 115 and the wearable device 10 through cables or wireless connection. As the information processing device 200, a microprocessor may be incorporated in the wearable device 10. Alternatively, the information processing device 200 may be, for example, a personal computer, a smartphone, or a tablet terminal independent of the wearable device 10. The sensor 115 may be connected to the wearable device 10 or the information processing device 200 by wiring or wirelessly.

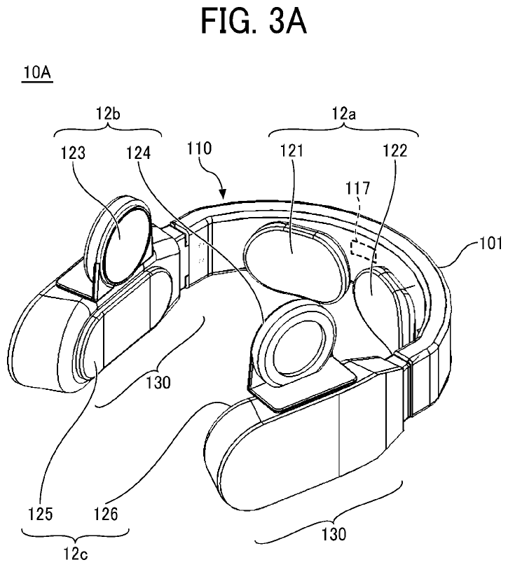

FIG. 2 illustrates the wearable device 10 worn by a user, according to the present embodiment. The wearable device 10 is worn on a body part of a user 5 where the user 5 can be efficiently aroused. The wearable device 10 according to the present embodiment is designed to be worn on a neck 51 of the user 5. Although the wearable device 10 has a U-shape surrounding a part of the periphery of the neck 51 of the user 5 in the example of FIG. 2, the wearable device 10 may have a C-shape. Alternatively, the wearable device 10 may have an O-shape surrounding the entire periphery of the neck 51 of the user 5. Other than such shapes to be worn on the neck 51, the shape and configuration of the wearable device 10 are variable to be worn on a shoulder, a waist, an upper arm, a lower arm, or any other appropriate part of the body of the user 5. The stimulator 12 applies one or more different types of stimuli to the user 5.

For example, a stimulus for increasing the level of arousal or concentration and a stimulus for decreasing the level of arousal (a stimulus for decreasing the state of arousal) are applied to appropriate parts of the neck 51. A thermal stimulus and an electrical stimulus may be applied simultaneously or alternately to adjust the level of arousal. Three or more types of stimuli may be applied simultaneously or separately. Instead of applying stimuli of different types separately, stimuli of the same type having different strengths may be applied. The level of arousal indicates the degree of arousal or relaxation of a user (target person). In the following description, the level of arousal may be referred to as a state of arousal, whereas the arousal may be referred to as tension or being nervous. In the evaluation of levels of arousal, a high level of arousal indicates that the user is more nervous than at a reference level of arousal, whereas a low level of arousal indicates that the user is more relaxed than at the reference level of arousal. According to the embodiments of the present disclosure, the reference level of arousal is, for example, a “target level of arousal” described later.

In a case where the sensor 115 is incorporated in the wearable device 10, the sensor 115 may be disposed on the inner side of the wearable device 10 at a position where the sensor 115 contacts the carotid artery below an car 52 of the user 5 or may be worn on an earlobe of the car 52 or the wrist of the user 5.

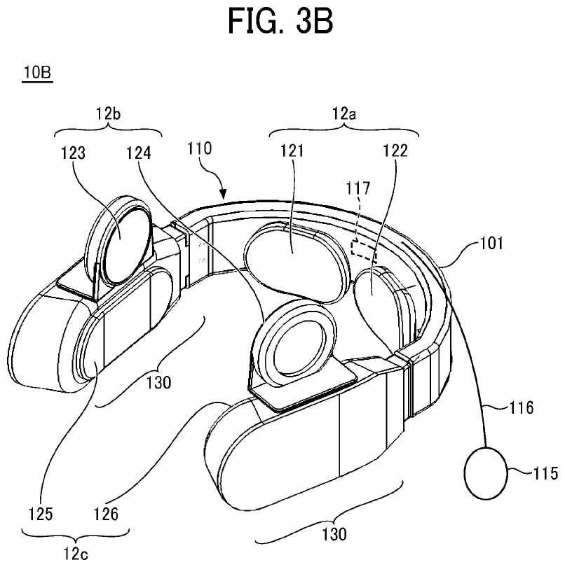

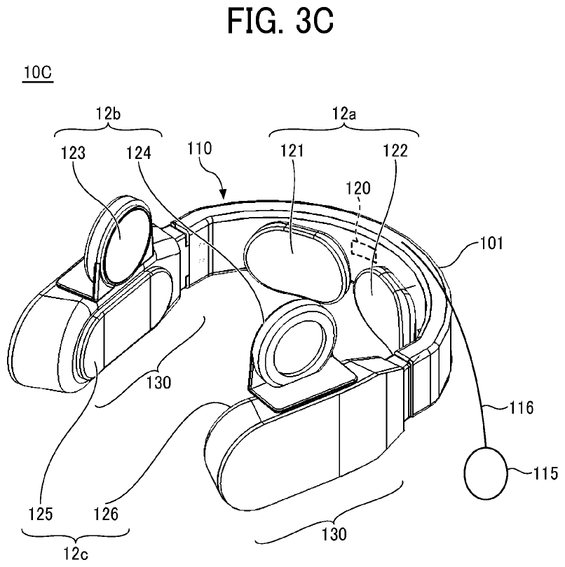

FIGS. 3A. 3B, and 3C illustrate different configurations of the wearable device 10. Specifically, FIG. 3A illustrates a wearable device 10A according to a first example of the present disclosure. The wearable device 10A includes a body 110 and stimulators 12a. 12b, and 12c on the body 110. The stimulators 12a, 12b, and 12c may be referred to simply as stimulator(s) 12 in the following description.

The wearable device 10A illustrated in FIG. 3A is independent of the sensor 115 and the information processing device 200 in the arousal state control system 1 of FIG. 1. The body 110 of the wearable device 10A is provided with a communication device 117. The communication device 117 receives a control signal indicating the result of analysis of biological information from the external information processing device 200. A part or all of the stimulators 12a, 12b, and 12c are driven according to the received control signals. The information processing device 200 may be any device provided that the device has a function of analyzing biological information acquired from the sensor 115 and transmitting the result of analysis to the wearable device 10. As described above, for example, a personal computer, a smartphone, or a tablet terminal may be used as the information processing device 200.

The stimulators 12a. 12b, and 12c generate different stimuli from each other. As an example, the stimulator 12a includes first electrodes 121 and 122 disposed on the inner side of a curved portion 101 of the U-shaped body 110. The first electrodes 121 and 122 come into contact with the back or nape of the neck 51 of the user 5 when the wearable device 10A is worn on the neck 51 of the user 5. The stimulator 12a applies a heat stimulus or a low-frequency electrical stimulus to the nape of the user 5 through the first electrodes 121 and 122. The number of first electrodes is not limited to two. Three or more first electrodes may be disposed along the inner face of the curved portion 101 of the body 110.

When the stimulator 12a generates heat, a heater disposed inside the stimulator 12a is activated according to the control signal received by the communication device 117 to impart heat through the first electrodes 121 and 122. When the stimulator 12a applies the low-frequency electrical stimulus, an oscillation circuit disposed inside the stimulator 12a is turned on to apply low-frequency vibration through the first electrodes 121 and 122. The low-frequency vibration provides electrical muscle stimulation (EMS). The heat or low-frequency vibration applied to the nape of the neck typically serves as a stimulus for relaxation, which relaxes the muscle tension. However, the heat or low-frequency vibration may also serve as a stimulus for arousal depending on individual differences or the stimulus strength.

The stimulator 12b is attached to arms 130 each extending on either side of the curved portion 101 of the body 110. The stimulator 12b includes second electrodes 123 and 124 each projecting from the arm 130 in a width (or height) direction of the body 110. When the wearable device 10A is worn on the neck 51 of the user 5, the second electrodes 123 and 124 come into contact with the skin surface around the carotid arteries below the cars 52 or the jaw of the user 5. The stimulator 12b applies a high-frequency electrical stimulus to the carotid arteries of the user 5 through the second electrodes 123 and 124. An oscillation circuit disposed inside the stimulator 12b is turned on to apply high-frequency vibration through the second electrodes 123 and 124. The high-frequency vibration typically serves as a stimulus for relaxation, which improves blood flow. However, the high-frequency vibration may also serve as a stimulus for arousal depending on individual differences or the stimulus strength.

The stimulator 12c is disposed on the inner side of the arms 130 each extending on either side of the curved portion 101 of the body 110. The stimulator 12c includes third electrodes 125 and 126 facing each other. When the wearable device 10A is worn on the neck 51 of the user 5, the third electrodes 125 and 126 come into contact with the skin surface around the carotid arteries along the neck of the user 5. The stimulator 12c applies a cooling stimulus or a heat stimulus to the carotid arteries of the user 5 through the third electrodes 125 and 126. When the stimulator 12e applies the cooling or heat stimulus, the cooling or heat stimulus is generated under the control of ON/OFF of the voltage for driving a Peltier element disposed inside the stimulator 12c and the polarity of the applied voltage. Appropriate cooling and warming of the carotid arteries typically acts in the direction of increasing the level of arousal. However, when the blood vessel is excessively expanded, a cool stimulus, which provides feelings of coolness, may relax the user.

FIG. 3B is a schematic diagram of a wearable device 10B according to a second example of the present disclosure. The wearable device 10B includes the sensor 115 connected to the communication device 117, in addition to the configuration of FIG. 3A. The wearable device 10B illustrated in FIG. 3B includes the sensor 115 and is independent of the information processing device 200 in the arousal state control system 1 of FIG. 1. In the example of FIG. 3B, the sensor 115 is connected to the body 110 through a cable 116 and is electrically connected to the communication device 117. Alternatively, the sensor 115 may be wirelessly connected to the communication device 117. In a case where the sensor 115 can directly communicate with the external information processing device 200 without the communication device 117, the configuration is as illustrated in FIG. 3A. The sensor 115 is a biological sensor such as a heart rate sensor, a pulse sensor, a temperature sensor, or a perspiration sensor. In a case where the sensor 115 is a heart rate sensor or a pulse sensor, the sensor 115 may be a type of sensor attachable to an earlobe or a fingertip of the user 5 or may be another type of sensor attachable to the chest of the user 5.

The communication device 117 transmits the biological information acquired by the sensor 115 to the external information processing device 200. The information processing device 200 analyzes the acquired biological information, determines a stimulus to be applied to the user 5, and transmits the determined stimulus to the wearable device 10B as a control signal. The wearable device 10B drives a part or all of the stimulators 12a. 12b, and 12c, based on the control signal received by the communication device 117. The configurations and functions of the stimulators 12a. 12b, and 12c on the body 110 are as described above with reference to FIG. 3A.

FIG. 3C is a schematic diagram of a wearable device 10C according to a third example of the present disclosure. The wearable device 10C includes a microprocessor 120 in the body 110 and the sensor 115 connected to the microprocessor 120. The wearable device 10C illustrated in FIG. 3C includes the sensor 115 and the information processing device 200 in the arousal state control system 1 of FIG. 1. Although the information processing device 200 is implemented by the microprocessor 120, the information processing device 200 may be implemented by a field programmable gate array (FPGA) or an application-specific integrated circuit (ASIC). In FIG. 3C, the sensor 115 is connected to the body 110 through the cable 116 and is connected to the microprocessor 120. Alternatively, the sensor 115 may be wirelessly connected to the microprocessor 120 with built-in communication functions.

The microprocessor 120 analyzes the biological information acquired from the sensor 115 and drives a part or all of the stimulators 12a, 12b, and 12c on the body 110. The configurations and functions of the stimulators 12a. 12b, and 12c are as described above with reference to FIG. 3A. Other than the configurations illustrated in FIGS. 3A, 3B, and 3C, the body 110 may be simply provided with the microprocessor 120 with wireless communication functions. In this case, the wearable device 10 and the information processing device 200 are integrated and independent of the sensor 115 in the arousal state control system 1 of FIG. 1. The configurations of the arousal state control system 1 and the wearable device 10 may be any configurations provided that the configurations of the arousal state control system 1 and the wearable device 10 allow control of the state of arousal of the user with one or more types of stimuli generated based on biological information acquired.

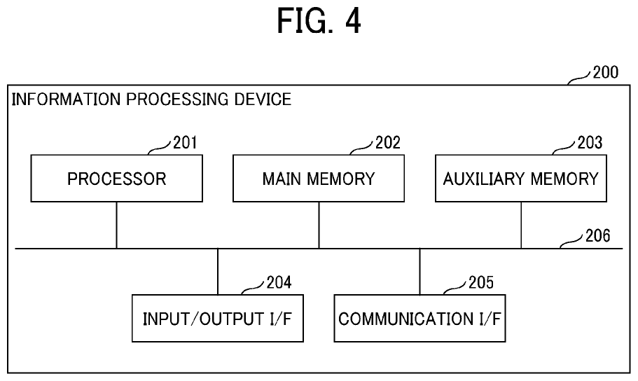

FIG. 4 is a block diagram illustrating a hardware configuration of the information processing device 200 according to the present embodiment. The information processing device 200 includes a processor 201, a main memory 202, an auxiliary memory 203, an input/output interface (I/F) 204, and a communication interface (I/F) 205, which are connected to each other through a system bus 206.

The processor 201, which is implemented by a central processing unit (CPU), executes control processing including various types of arithmetic processing and implements the functions of the stimulation control unit 210. The control processing executed by the processor 201 includes the acquisition of biological information, analysis of the biological information, the determination of a stimulus, the acquisition of a target level of arousal set by the user, and the acquisition of a subjective evaluation input by the user.

The determination of a stimulus includes the determination of, for example, the stimulus type, a combination of stimuli, the duration of stimulation, and the stimulus strength, and correction or change of the determined stimulus type, the determined combination of stimuli, the determined duration of stimulation, and the determined stimulus strength. As will be described later, for example, the type of stimulus to be applied, the duration of stimulation, and the target level of arousal corresponding to work may be corrected based on a change in the difference between the target level of arousal and the level of arousal calculated from the biological information and the subjective evaluation of performance.

The main memory 202 includes a read-only memory (ROM) that stores programs used for the operation of the processor 201 and a random access memory (RAM) that is used as a work area of the processor 201. The auxiliary memory 203 includes a storage device such as a hard disk drive (HDD) or a solid-state drive (SSD). The auxiliary memory 203 stores, in addition to various programs and parameter information to be used to start the programs, information and parameters used for arousal state control.

The input/output interface 204 connects input/output devices such as a display, a touch panel, a speaker, an earphone, a microphone, and a keyboard to the information processing device 200. The communication interface 205 enables communication between the information processing device 200 and an external device through, for example, a public communication network, a local area network (LAN), or a short-range communication standard. The communication between the information processing device 200 and the sensor 115 or the wearable device 10 may be performed according to a short-range wireless communication standard.

A description is given below of a functional configuration of the stimulation control unit 210 according to the present embodiment.

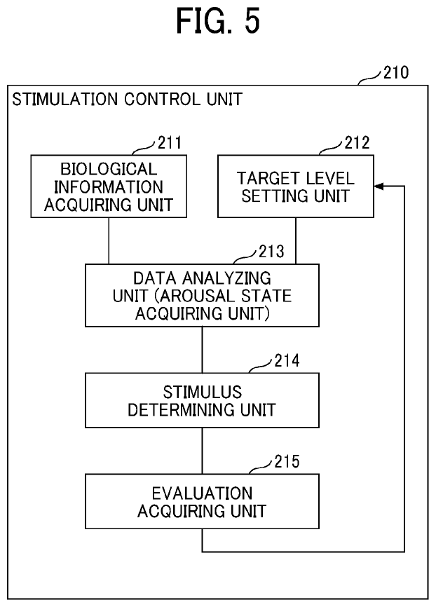

FIG. 5 is a functional block diagram of the stimulation control unit 210 according to the present embodiment. The stimulation control unit 210 is implemented by the processor 201. The stimulation control unit 210 includes a biological information acquiring unit 211, a target level setting unit 212, a data analyzing unit 213, a stimulus determining unit 214, and an evaluation acquiring unit 215.

The biological information acquiring unit 211 acquires biological information of a user who wears the wearable device 10. The biological information may be directly acquired from the sensor 115 worn by the user as described above. Alternatively, the biological information may be acquired from the wearable device 10.

The target level setting unit 212 acquires the setting of a target state of arousal input by the user. The target state of arousal indicates a target degree of arousal or tension when the user performs work. The target state of arousal may be represented by a numerical value such as a breathing rate, a perspiration level, a heart rate, or the number of blinks. In this sense, the target state of arousal is referred to as a “target level of arousal.” In the present embodiment, a parameter indicating the level of arousal is a ratio (LF/HF) between a low frequency (LF) component and a high frequency (HF) component included in a power spectrum of variation in heart rate, which is obtained by analyzing the heart rate. In this case, the target level of arousal is set as a target LF/HF value.

The LF component is a frequency band in a range of 0.004 hertz (Hz) to 0.150 Hz in the power spectrum of variation in heart rate. The LF component is considered to reflect the activities of both the sympathetic nerves and the parasympathetic nerves. The HF component is a frequency band in a range of 0.150 Hz to 0.400 Hz in the power spectrum of variation in heart rate. The HF component is considered to reflect the activity of parasympathetic nerves. The ratio of LF to HF (i.e., LF/HF) indicates which of the sympathetic nerves and the parasympathetic nerves are dominant to what extent. When the user is aroused or nervous, the sympathetic nerves are dominant over the parasympathetic nerves. In other words, the LF/HF value is relatively large. When the user is relaxed, the parasympathetic nerves are dominant over the sympathetic nerves. In other words, the LF/HF value is relatively small.

The data analyzing unit 213 analyzes the biological information to determine the level of arousal of the user. The data analyzing unit 213 may be referred to as an arousal state acquiring unit in the following description. When the ratio of LF to HF (i.e., LF/HF) is used as a parameter indicating the level of arousal, the data analyzing unit 213 extracts the LF component and the HF component from the acquired spectrum of variation in heart rate data, to calculate the ratio of LF to HF (i.e., LF/HF). The data analyzing unit 213 also refers to the target level of arousal set by the user and specifies the difference between the calculated LF/HF value and the target level of arousal. When the difference between the current LF/HF value and the target level of arousal exceeds an allowable range, the data analyzing unit 213 detects that the user is excessively relaxed or excessively nervous. The data analyzing unit 213 monitors the direction of change in the difference.

The stimulus determining unit 214 determines a stimulus to be applied to the user, based on the result of analysis conducted by the data analyzing unit 213. When the data analyzing unit 213 determines that the user is excessively relaxed, based on the difference between the calculated LF/HF value and the target level of arousal, the stimulus determining unit 214 selects a stimulus for increasing the level of arousal. By contrast, when the data analyzing unit 213 determines that the user is excessively nervous, the stimulus determining unit 214 selects a stimulus for decreasing the level of arousal. Since what type of stimulus is effective for increasing or decreasing the level of arousal varies depending on the user, the tendency of the user may be measured in advance to allow the stimulus determining unit 214 to determine stimuli for arousal and relaxation effective for the user.

For a user who can be relaxed from a nervous state when the carotid arteries of the user are cooled, the stimulus determining unit 214 selects a cool stimulus when the user is excessively nervous. For a user who is aroused by low-frequency electrical stimulation, the stimulus determining unit 214 selects the low-frequency electrical stimulus when the user is excessively relaxed. By contrast, for a user who is aroused from a relaxed state when the carotid arteries of the user are cooled, the stimulus determining unit 214 selects the cool stimulus when the user is excessively relaxed. For a user who is relaxed by low-frequency electrical stimulation, the stimulus determining unit 214 selects the low-frequency electrical stimulus when the user is excessively nervous.

As the first stimulus, a stimulus that is typically considered to be effective for arousal or relaxation may be selected by default. Alternatively, a stimulus may be selected at random from the stimulators 12 of the wearable device 10. By repeatedly acquiring the biological information while the user performs work and determining the state of arousal based on the change in the biological information with respect to the target level of arousal, the stimulation control unit 210 can correct or change the initially selected stimulus as appropriate and lead the level of arousal of the user to the target level of arousal. The correction or change of the stimulus will be described later.

The evaluation acquiring unit 215 acquires an evaluation as to whether the wearable device 10 has improved performance of the user wearing the wearable device 10. The evaluation may be a subjective evaluation that the user inputs to the information processing device 200 after finishing the work. Alternatively, the evaluation may be automatically determined by the data analyzing unit 213 based on the degree of approaching the target level of arousal or the time to reach the target level of arousal.

The stimulation control unit 210 determines different types of stimuli based on the biological information of the user, to control the state of arousal of the user as appropriate. The stimulation control unit 210 corrects or changes, for example, the stimulus type, the duration of stimulation, and the stimulus strength, based on a detected difference between the current level of arousal and the target level of arousal, to lead the level of arousal to an appropriate level of arousal. The feedback on the result of the evaluation of performance allows review of the target level of arousal for each user.

A description is given below of an arousal state control process according to the present embodiment.

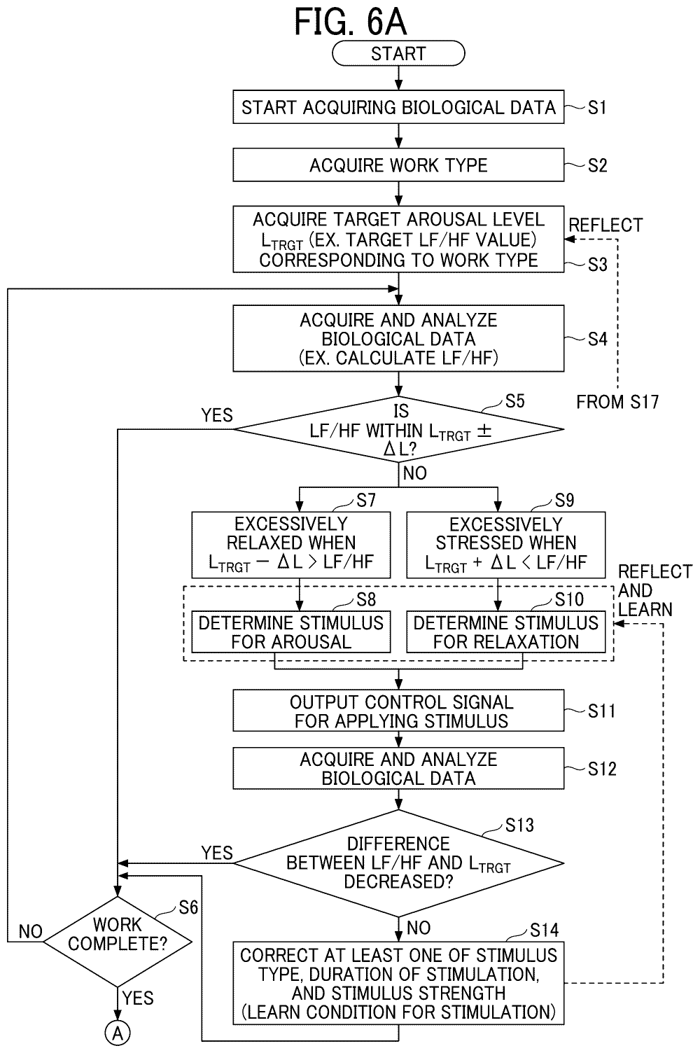

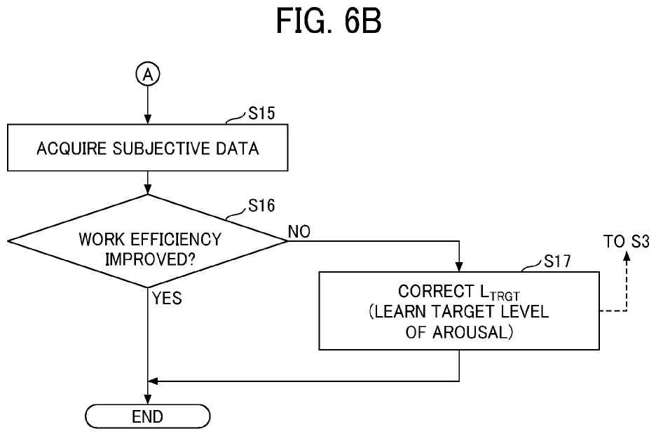

FIG. 6A is a flowchart of arousal state control executed by the stimulation control unit 210. i.e., the processor 201 according to the present embodiment. FIG. 6B is a continuation of the flowchart of FIG. 6A. Specifically, the process illustrated in FIG. 6B continues from a node A illustrated in FIG. 6A. In step S1, the stimulation control unit 210 starts acquiring biological information from the sensor 115. When a user wears the wearable device 10 and activates a predetermined application of the information processing device 200, the stimulation control unit 210 starts acquiring the biological information.

In step S2, the stimulation control unit 210 acquires the work type. In step S3, the stimulation control unit 210 acquires, as a target level of arousal, a target arousal level L_{TRGT} corresponding to the work type. The work type and the target arousal level L_{TRGT} are input to the information processing device 200 by the user. The work type represents the degree of intellectual work in which the thought of the user is involved. The work type is represented by, for example, simple work, normal work, and complicated work. A scheduled work time may be input together with the work type. The target arousal level L_{TRGT} indicates a state of arousal required or desired for the scheduled work.

In the present embodiment, a target LF/HF value as the target level of arousal or a range of target LF/HF values is input by the user. When the ratio of LF to HF (i.e., LF/HF) is used, the target level of arousal may be set in a range not less than 1.5 and not greater than 5.0 as appropriate for the work type. For simple work, the target arousal level L_{TRGT} may be set to be slightly higher so as not to cause drowsiness. For complicated intellectual work, the target arousal level L_{TRGT} may be set slightly lower so that the user is not excessively stressed. The order of steps S2 and S3 may be reversed. Alternatively, the stimulation control unit 210 may simultaneously perform the operations in steps S2 and S3.

In step S4, the stimulation control unit 210 acquires and analyzes the biological data (i.e., biological information). The stimulation control unit 210 analyzes the acquired biological information (e.g., heart rate data) at predetermined time intervals and extracts the LF component and the HF component included in the variation in heart rate data, to calculate the ratio of LF to HF (i.e., LF/HF). For example, the stimulation control unit 210 analyzes the heart rate data for five minutes and calculates the LF/HF value. The processing is updated every minute. However, the present disclosure is not limited to the aforementioned way. The data accumulation time and the time interval for updating may be set as appropriate. For example, the time intervals for data analysis and the calculated LF/HF values are as follows.

| Time interval | LF/HF |
|---|---|
| 10:00-10:05 | 2.0 |
| 10:01-10:06 | 1.4 |
| 10:02-10:07 | 1.8 |
| 10:03-10:08 | 1.2 |

Subsequently, the LF/HF value is calculated for each analysis section, which is a time interval of 5 minutes and shifted every minute.

In step S5, the stimulation control unit 210 determines whether the LF/HF value calculated for each analysis section is within a predetermined range (L_{TRGT}±ΔL) in the vicinity of the target arousal level L_{TRGT}. When the calculated LF/HF value is within the range of L_{TRGT}±ΔL (YES in step S5), the user is in an appropriate state of arousal for the work type. In step S6, the stimulation control unit 210 determines whether the work is complete. When the work is not complete (NO in step S6), the stimulation control unit 210 repeats acquiring and analyzing the biological data (in step S4) and checking the state of arousal (in step S5) until the work is complete (YES in step S6).

When the calculated LF/HF value is not within the range of L_{TRGT}±ΔL (NO in step S5), the state of arousal of the user is away from the target level of arousal set according to the work type. In step S7, the stimulation control unit 210 determines that the user is excessively relaxed for the current work when the calculated LF/HF value is smaller than the allowable range (L_{TRGT}−ΔL>calculated value). In this case, in step S8, the stimulation control unit 210 selects a stimulus for increasing the level of arousal, that is, a stimulus for arousal. When the user is effectively aroused by low-frequency electrical stimulation, the stimulator 12a is driven to apply the low-frequency electrical stimulus. The stimulation control unit 210 may determine the duration of stimulation, together with the determination of the stimulus.

In step S9, the stimulation control unit 210 determines that the user is excessively nervous (or stressed) for the current work when the calculated LF/HF value is greater than the allowable range (L_{TRGT}+ΔL<calculated value). In this case, in step S10, the stimulation control unit 210 selects a stimulus for decreasing the level of arousal, that is, a stimulus for relaxation. When the user is relaxed by cool stimulation to the carotid arteries, the stimulator 12c is driven to cool the nape of the user as appropriate. Alternatively, the stimulator 12a may be driven to apply a heat stimulus to the nape of the user to warm the back of the neck. Both the stimulators 12a and 12c may be driven. The stimulation control unit 210 may determine the duration of stimulation and the stimulus strength, together with the determination of the stimulus for relaxation.

In step S11, the stimulation control unit 210 outputs, to the stimulator 12, a control signal for applying the stimulus determined in step S8 or S10. Based on the control signal, the stimulator 12 generates and applies the stimulus.

In step S12, the stimulation control unit 210 continues to acquire and analyze the biological data. In step S13, the stimulation control unit 210 determines whether the difference between the calculated LF/HF value and the target level of arousal is changing in a decreasing direction. When the difference between the calculated LF/HF value and the target level of arousal decreases (YES in step S13), the selected stimulus is appropriate. Thus, the stimulation control unit 210 repeats acquiring and analyzing the biological data (in step S4) and checking the state of arousal (in step S5) until the work is complete (YES in step S6).

By contrast, when the difference between the calculated LF/HF value and the target level of arousal does not decrease (NO in step S13), the selected stimulus is not appropriate. Thus, in step S14, the stimulation control unit 210 corrects or changes at least one of the stimulus type, the duration of stimulation, and the stimulus strength. This correction is reflected in the selection of the stimulus for arousal in step S8 and the selection of the stimulus for relaxation in step S10. Thus, the determination of the stimulus is learned. Subsequently, the stimulation control unit 210 repeats acquiring and analyzing the biological data (in step S4) and checking the state of arousal (in step S5) until the work is complete (YES in step S6).

When the work is complete (YES in step S6), in step S15, the stimulation control unit 210 acquires an evaluation on the improvement of performance. The evaluation may be a subjective evaluation of the user indicating whether the stimulation has improved the user's work efficiency or performance. In step S16, for example, the stimulation control unit 210 determines whether the subjective evaluation indicates an improvement in work efficiency. When the subjective evaluation indicates an improvement in work efficiency (YES in step S16), the stimulation is successful for a specific work type. In this case, the process ends while maintaining the set target level of arousal and the selected stimulus type and duration of stimulation.

By contrast, when the work efficiency is not improved despite the correction of the stimulus (NO in step S16), the target level of arousal set for the specific work type may be inappropriate. In step S17, the stimulation control unit 210 corrects the target arousal level L_{TRGT}. Thus, the process ends. The correction of the target arousal level L_{TRGT} is reflected in the setting of the target arousal level L_{TRGT} in step S3 and learned.

In the arousal state control illustrated in FIGS. 6A and 6B, the stimulation control unit 210 may determine the level of arousal at predetermined time intervals in step S5. For example, the stimulation control unit 210 may determine the state of arousal of the user at intervals of 10 minutes or 15 minutes. With this arousal state control method, the stimulation control unit 210 can control the state of arousal as appropriate for the work performed by the user.

A description is given below of evaluations of arousal state control according to the present embodiment.

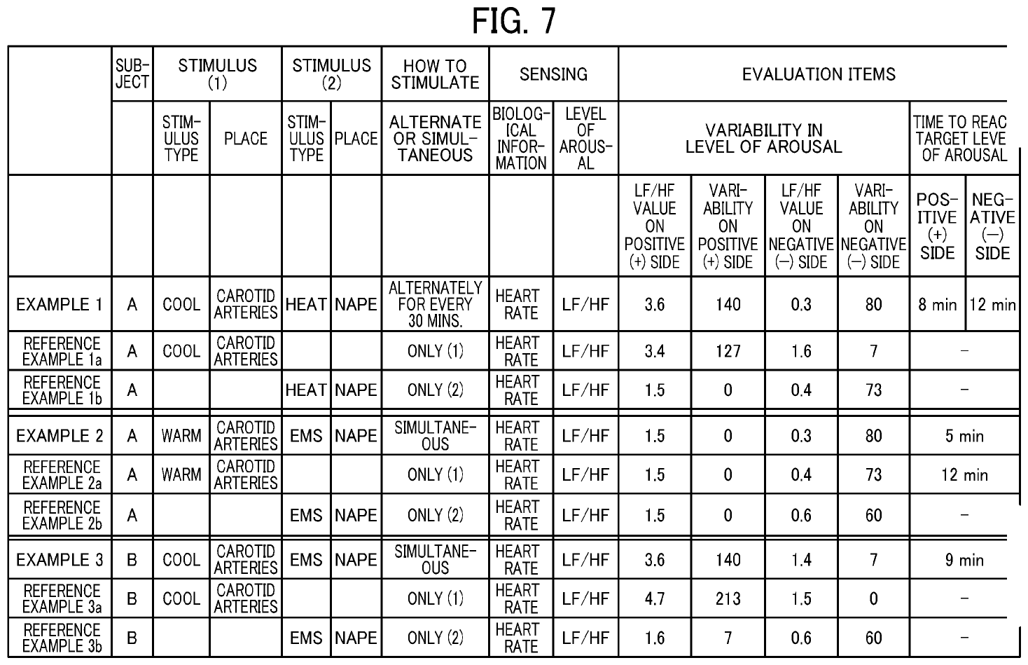

FIG. 7 is a table illustrating the result of evaluations of arousal state control. Each of a user A and a user B wears the wearable device 10. In the evaluations illustrated in FIG. 7, both the users A and B are adult males. The wearable device 10 is applicable to any user regardless of the gender or age of the user. Under the control of the information processing device 200, one or more types of stimuli are applied to each of the users A and B. In all evaluations, the heart rate data is acquired as biological information, whereas the LF/HF value is calculated as a parameter indicating a level of arousal. The LF/HF value is calculated by analyzing the heart rate data for 5 minutes and is updated every minute.

The evaluation items are the variability in the level of arousal and the time to reach the target level of arousal. The variability in the level of arousal indicates variation on the positive (+) side, that is, variation in an overstressed state in which the LF/HF value is higher than the target level of arousal, and variation on the negative (−) side, that is, variation in an overrelaxed state in which the LF/HF value is lower than the target level of arousal. The time to reach the target level of arousal may be also indicated for the positive and negative sides.

A variability R_{CHN} (%), as variability in the level of arousal, is calculated as follows:

$${R}_{CHN} = 100 \times ( A F T - B FR ) / BFR ,$$

where BFR represents the LF/HF values for 15 points immediately before stimulation and AFT represents the LF/HF values for 15 points from the maximum IF/HF value during stimulation.

The time to reach the target level of arousal is a time when the average of variabilities of three points reaches a range of 100±10%, which is a target variability. When the target variability is not reached by continuous stimulation for 30 minutes or when the LF/HF value swings upward, the variability R_{CHN} in the level of arousal is evaluated as 0 (%).

A description is given below of Example 1 illustrated in FIG. 7.

In Example 1, Stimuli (1) and (2) of different types are applied to the carotid arteries of the user A and the nape of the neck of the user A, respectively. Specifically, Stimulus (1) of Example 1 is a cool stimulus applied to the carotid arteries, whereas Stimulus (2) of Example 1 is a heat stimulus applied to the nape of the neck. The stimulators 12c and 12a of the wearable device 10 are alternately driven to alternately apply Stimulus (1) and Stimulus (2) for 30 minutes each. When Stimulus (1) and Stimulus (2) are alternately applied, the LF/HF value on the positive (+) side (i.e., in the overstressed state) is 3.6, the variability R_{CHN} is 140%, and the time to reach the target level of arousal (i.e., the target LF/HF value) is 8 minutes. The LF/HF value on the negative (−) side (i.e., in the overrelaxed state) is 0.3, whereas the variability R_{CHN} is 80%. The time to reach the target level of arousal is 12 minutes. The user may be aroused more efficiently by different types of stimuli than by a single stimulus. In this case, both the control for increasing the level of arousal and the control for decreasing the level of arousal are achieved. As a result, the target level of arousal is reached in about 10 minutes.

A description is given below of Reference Example 1a illustrated in FIG. 7.

Reference Example 1a is the result of evaluation when Stimulus (1) alone, of the stimuli applied in Example 1, is applied to the carotid arteries. The LF/HF value on the positive (+) side (i.e., in the overstressed state) is 3.4, whereas the variability R_{CHN} is 120%. The LF/HF value on the negative (−) side (i.e., in the overrelaxed state) is 1.6, whereas the variability R_{CHN} is 7%. Reaching the target level of arousal may be difficult when Stimulus (1) alone is continuously applied for 30 minutes. In this example, although cool stimulation to the carotid arteries relaxes the user A to some extent, the target level of arousal is not reached. In other words, the cool stimulus is inappropriate for the user A as a stimulus for increasing the level of arousal. Selecting another stimulus may increase the level of arousal of the user A.

A description is given below of Reference Example 1b illustrated in FIG. 7.

Reference Example 1b is the result of evaluation when Stimulus (2) alone, of the stimuli applied in Example 1, is applied to the nape of the neck. The LF/HF value on the positive (+) side (i.e., in the overstressed state) is 1.4, whereas the variability R_{CHN} is 0%. The LF/HF value on the negative (−) side (i.e., in the overrelaxed state) is 0.4, whereas the variability R_{CHN} is 73%. When Stimulus (2) alone is continuously applied for 30 minutes, the arousal effect is higher as compared with a case where Stimulus (1) is applied. For the user A, the heat stimulation to the nape of the neck increases the level of arousal to some extent. In other words, the heat stimulation can act in the direction of reducing tension.

A description is given below of Example 2 illustrated in FIG. 7.

In Example 2. Stimuli (1) and (2) of different types are applied to the carotid arteries of the user A and the nape of the neck of the user A, respectively. Specifically, Stimulus (1) of Example 2 is a warm stimulus applied to the carotid arteries, whereas Stimulus (2) is (low-frequency) EMS to the nape of the neck. The stimulators 12c and 12a of the wearable device 10 are driven at the same time to apply Stimulus (1) and Stimulus (2) at the same time. When Stimulus (1) and Stimulus (2) are applied at the same time, the LF/HF value on the negative (−) side (i.e., in the overrelaxed state) is 0.3, the variability R_{CHN} is 80%, and the time to reach the target level of arousal is 5 minutes. On the positive (+) side, the LF/HF value is as low as 1.5, and this value hardly fluctuates. The target level of arousal is quickly reached by the application of different stimuli at the same time.

A description is given below of Reference Example 2a illustrated in FIG. 7.

Reference Example 2a is the result of evaluation when Stimulus (1) alone, of the stimuli applied in Example 2, is applied to the carotid arteries. The LF/HF value on the negative (−) side (i.e., in the overrelaxed state) is 0.4, the variability R_{CHN} is 73%, and the time to reach the target level of arousal is 12 minutes. The LF/HF value on the positive (+) side is as low as 1.5, and this value hardly fluctuates. Reference Example 2a is included in one of the specific examples of embodiments of the present disclosure. In Reference Example 2a, the level of arousal is controlled toward the target level of arousal by the application of the warm stimulus of Stimulus (1) alone to the carotid arteries. However, it takes more time to reach the target level of arousal than in Example 2 in which the EMS of Stimulus (2) is applied in combination with Stimulus (1).

A description is given below of Reference Example 2b illustrated in FIG. 7.

Reference Example 2b is the result of evaluation when Stimulus (2) alone, of the stimuli applied in Example 2, is applied to the nape of the neck. The LF/HF value on the negative (−) side (i.e., in the overrelaxed state) is 0.6, whereas the variability R_{CHN} is 60%. Although the application of Stimulus (2) alone can increase the level of arousal to some extent, the target level of arousal is not reached by the continuous application of Stimulus (2) for 30 minutes. The LF/HF value on the positive (+) side is as low as 1.5, and this value hardly fluctuates.

A description is given below of Example 3 illustrated in FIG. 7.

In Example 3, Stimuli (1) and (2) of different types are applied to the carotid arteries of the user B and the nape of the neck of the user B, respectively. Specifically. Stimulus (1) of Example 3 is the cool stimulus applied to the carotid arteries, whereas Stimulus (2) is the (low-frequency) EMS to the nape of the neck. The stimulators 12c and 12a of the wearable device 10 are driven at the same time to apply Stimulus (1) and Stimulus (2) at the same time. When Stimulus (1) and Stimulus (2) are applied at the same time, the LF/HF value on the positive (+) side (i.e., in the overstressed state) is 3.6, whereas the variability R_{CHN} is 140%. The LF/HF value on the negative (−) side (i.e., in the overrelaxed state) is 1.4, whereas the variability R_{CHN} is 7%. The time to reach the target level of arousal is 9 minutes. A slight fluctuation of the LF/HF value on the negative (−) side may indicate that Stimulus (1) acts on relaxation while Stimulus (2) finely adjusts the level of arousal in the direction of slightly increasing the level of arousal. Application of different stimuli enables both the control in the direction of decreasing the level of arousal and the control in the direction of increasing the level of arousal. As a result, the target level of arousal is easily reached.

A description is given below of Reference Example 3a illustrated in FIG. 7. Reference Example 3a is the result of evaluation when the cool stimulus of Stimulus (1) alone, of the stimuli applied in Example 3, is applied to the carotid arteries. The LF/HF value on the positive (+) side (i.e., in the overstressed state) is 4.7, whereas the variability R_{CHN} is 213%. The LF/HF value on the negative (−) side (i.e., in the overrelaxed state) is 1.4, whereas the variability R_{CHN} is 0%. For the user B, the strength of Stimulus (1) is too strong and the LF/HF value swings upward, resulting in a failure of evaluation. In this case, by combining Stimulus (2) in the direction of increasing the level of arousal with Stimulus (1) as in Example 3, the level of arousal can be controlled to a desired level of arousal.

A description is given below of Reference Example 3b illustrated in FIG. 7.

Reference Example 3b is the result of evaluation when Stimulus (2) alone, of the stimuli applied in Example 3, is applied to the nape of the neck. The LF/HF value on the negative (−) side (i.e., in the overrelaxed state) is 0.6, whereas the variability R_{CHN} is 60%. The target level of arousal is not reached by the continuous application of Stimulus (2) alone for 30 minutes. The LF/HF value on the positive (+) side is as low as 1.5, and this value hardly fluctuates. For this user, the target level of arousal can be achieved more effectively by combining a plurality of types of stimuli than by the EMS alone to the nape of the neck.

The result illustrated in FIG. 7 indicates that the same stimulus can be a stimulus for relaxation or a stimulus for arousal, depending on the user. The tendency of the user may be acquired in advance to classify multiple types of stimuli that can be applied by the wearable device 10 into a stimulus for relaxation or a stimulus for arousal and record the classified multiple types of stimuli in a built-in memory of the information processing device 200 or the microprocessor 120. When a high relaxation effect or an appropriate arousal effect can be obtained by combining two or more different types of stimuli, the combination of different types of stimuli may be recorded in the built-in memory of the information processing device 200 or the microprocessor 120. For example, when the relaxation effect or the arousal effect is excessive with a single application of the warm stimulus to the carotid arteries or the low-frequency electrical stimulus to the nape of the neck, simultaneous application of the warm stimulus to the carotid arteries and the low-frequency electrical stimulus to the nape of the neck can finely adjust the state of arousal to a desired state of arousal.

When the arousal state control method according to the embodiments of the present disclosure is implemented by a computer program, an arousal state control program is installed in the information processing device 200. The arousal state control program causes the processor 201 to execute: a procedure (a) of acquiring biological information of a user; a procedure (b) of analyzing the acquired biological information and determining one or more types of stimuli to control a state of arousal of the user; and a procedure (c) of notifying a stimulator of the determined one or more types of stimuli.

Although the arousal state control has been described above based on the specific embodiments or examples of the present disclosure, various modifications may be made within the scope of the technical idea of the present invention. In a case where the sensor 115, the communication device 117, or the microprocessor 120 is incorporated in the wearable device 10, the sensor 115, the communication device 117, or the microprocessor 120 may be connected to or disposed at an appropriate position of the arm 130, instead of being connected to or disposed at the curved portion 101 of the body 110. The biological information acquired by the sensor 115 is not limited to heart rate data. Other biological data such as a breathing rate, a sweating state, or the number of blinks may be detected to determine whether the user is nervous or relaxed. In addition to or instead of the thermal stimulus and the electrical stimulus, an aroma stimulus, a light stimulus, or a combination of these stimuli may be applied. Although the wearable device 10 to be worn on the neck as illustrated in FIG. 2 does not interfere with the work of the user and is effective in controlling the state of arousal, the wearable device of one or more embodiments of the present disclosure is not limited to a wearable device to be worn on the neck. The wearable device may be attached to any part of the body other than the neck, such as a wrist, an earlobe, a fingertip, a forearm, a thigh, or the back of a shoulder, provided that biological information such as a pulse, a body temperature, or a sweating state can be measured and a stimulus can be applied to the part.

A description is given below of wearable devices according to other examples of the present disclosure.

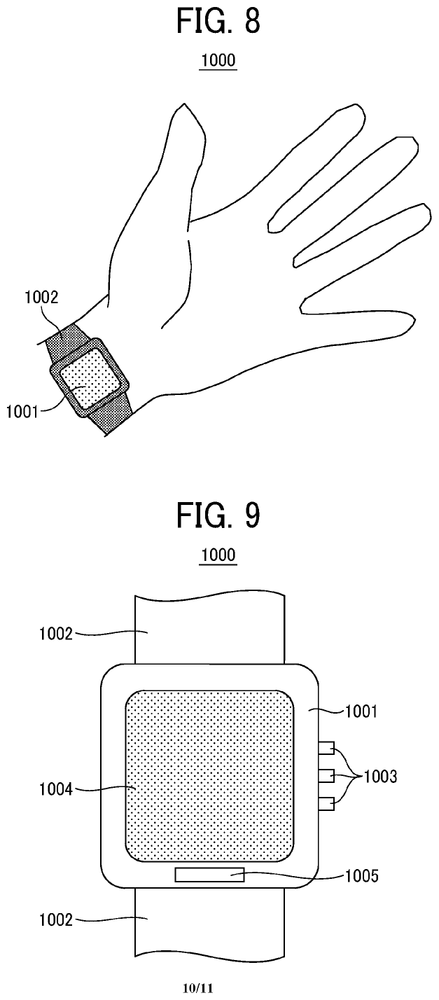

FIG. 8 is a diagram of a wearable device 1000 according to another example of the present disclosure. FIG. 9 is a diagram illustrating a configuration of a skin-contact side of the wearable device 1000 of FIG. 8. The skin-contact side of the wearable device 1000 contacts the skin of a user. The wearable device 1000 can be worn on a wrist. The wearable device 1000 includes a body 1001 and an attaching unit 1002. As illustrated in FIG. 9, the body 1001 includes an operation unit 1003, a stimulator 1004, and a sensor 1005. The stimulator 1004 is positioned to contact, for example, the inner side of a wrist. The sensor 1005 is disposed at a position where biological information can be easily acquired, for example, at the inner side of the wrist or at a position where the sensor 1005 can contact an artery. The operation unit 1003 may be disposed on a side of the body 1001. For example, the operation unit 1003 allows the user to turn on or off the power and adjust the stimulus strength. The communication device 117 (see FIGS. 3A and 3B) or the microprocessor 120 (see FIG. 3C) is incorporated in the body 1001 to transmit the acquired biological information to a remote processor or analyze the acquired biological information inside the wearable device 1000. Although FIG. 9 illustrates the single stimulator 1004, the number of stimulators is not limited to one. For example, another stimulator may be disposed on the same surface of the body 1001 on which the stimulator 1004 is disposed, to apply a plurality of types of stimuli such as a thermal stimulus and an electrical stimulus.

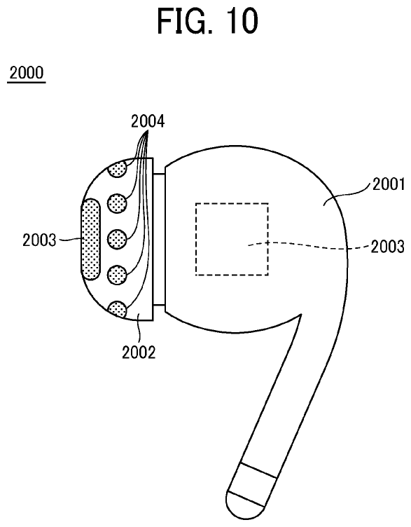

FIG. 10 is a diagram of a wearable device 2000 according to yet another example of the present disclosure. The wearable device 2000 can be worn on an car. The wearable device 2000 includes a body 2001, an attaching unit 2002, a stimulator 2003, and a sensor 2004. The body 2001 incorporates the communication device 117 or the microprocessor 120. The wearable device 2000 may incorporate a speaker as the stimulator 2003. The speaker can apply a sound stimulus to the user. The attaching unit 2002 has an earlobe-contact-face through which the attaching unit 2002 contacts an earlobe. For example, in a case where the stimulator 2003 applies a thermal stimulus, the stimulator 2003 is preferably positioned on the earlobe-contact-face of the attaching unit 2002 to easily contact the car. Similarly, the sensor 2004 is preferably disposed at a position on the attaching unit 2002 where the sensor 2004 can easily contact the car (earlobe).

Each of the wearable devices 1000 and 2000 can apply stimuli of different types or stimuli of the same type having different strengths to a part of the user in contact with the corresponding wearable device, based on the biological information of the user.

A description is given below of several aspects of the present disclosure.

According to a first aspect, an arousal state control system includes a sensor, a stimulation control unit, and a stimulator. The sensor acquires biological information of a user.

The stimulation control unit determines, based on the biological information, one or more types of stimuli to control a state of arousal. The stimulator applies the one or more types of stimuli determined by the stimulation control unit to the user.

According to a second aspect, in the arousal state control system of the first aspect, the stimulation control unit includes an arousal state acquiring unit that analyzes the biological information to acquire the state of arousal of the user. The stimulation control unit selects a stimulus that increases the state of arousal of the user in a case where the state of arousal of the user is lower than a target state of arousal. The stimulation control unit selects a stimulus that decreases the state of arousal of the user in a case where the state of arousal of the user is higher than the target state of arousal.

According to a third aspect, in the arousal state control system of the first or second aspect, the stimulation control unit determines, based on the biological information, a first stimulus that increases the state of arousal of the user and a second stimulus that decreases the state of arousal of the user. The stimulator simultaneously or alternately applies the first stimulus and the second stimulus.

According to a fourth aspect, in the arousal control system of any one of the first to third aspects, the stimulation control unit acquires a setting of a target state of arousal according to a work type and determines the one or more types of stimuli based on a change in the biological information with respect to the setting of the target state of arousal.

According to a fifth aspect, in the arousal control system of any one of the first to third aspects, the stimulation control unit acquires a setting of a target state of arousal according to a work type, and corrects at least one of a stimulus type, a duration of stimulation, and a stimulus strength, based on a change in the biological information with respect to the setting of the target state of arousal.

According to a sixth aspect, in the arousal control system of any one of the first to fifth aspects, the stimulation control unit determines two or more types of thermal stimuli or electrical stimuli based on the biological information. The stimulator simultaneously or alternately applies the two or more types of thermal stimuli or electrical stimuli. According to a seventh aspect, the arousal control system of any one of the first to sixth aspects, the stimulator applies the one or more types of stimuli to the neck of the user. According to an eighth aspect, a wearable device includes a body, a first stimulation unit, and a second stimulation unit. The first stimulation unit is disposed at a first portion of the body to apply a first stimulus. The second stimulation unit is disposed at a second portion of the body to apply a second stimulus different from the first stimulus. One or both of the first stimulation unit and the second stimulation unit are driven based on biological information of a user wearing the body.

According to a ninth aspect, the wearable device of the eighth aspect further includes a sensor and a communication device. The sensor acquires the biological information. The communication device transmits the biological information to an information processing device and receives, from the information processing device, a control signal that drives one or both of the first stimulation unit and the second stimulation unit.

According to a tenth aspect, the wearable device of the eighth aspect further includes a processor that acquires the biological information from a sensor and analyzes the biological information. One or both of the first stimulation unit and the second stimulation unit are driven based on a result of analysis of the biological information by the processor.

According to an eleventh aspect, the wearable device of the eighth aspect further includes a processor that analyzes the biological information and a biological sensor communicably connected to the processor. The processor analyzes the biological information based on an output of the biological sensor. One or both of the first stimulation unit and the second stimulation unit are driven based on a result of analysis of the biological information by the processor.

According to a twelfth aspect, an arousal state control method includes acquiring biological information of a user, analyzing the biological information with an information processing device, determining, with the information processing device, one or more types of stimuli to control a state of arousal of the user, and applying the determined one or more types of stimuli to the user.

According to a thirteenth aspect, in the arousal state control method of the twelfth aspect, the analyzing includes analyzing the biological information to acquire the state of arousal of the user with the information processing device. The determining includes selecting, with the information processing device, a stimulus that increases the state of arousal in a case where the state of arousal is lower than a target state of arousal, and selecting, with the information processing device, a stimulus that decreases the state of arousal in a case where the state of arousal is higher than the target state of arousal.

According to a fourteenth aspect, in the arousal state control method of the twelfth aspect, the applying includes simultaneously or alternately applying, to the user with a stimulator, a first stimulus that increases the state of arousal of the user and a second stimulus that decreases the state of arousal of the user, based on the determined one or more types of stimuli.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The present invention can be implemented in any convenient form, for example using dedicated hardware, or a mixture of dedicated hardware and software. The present invention may be implemented as computer software implemented by one or more networked processing apparatuses. The processing apparatuses include any suitably programmed apparatuses such as a general purpose computer, a personal digital assistant, a Wireless Application Protocol (WAP) or third-generation (3G)-compliant mobile telephone, and so on. Since the present invention can be implemented as software, each and every aspect of the present invention thus encompasses computer software implementable on a programmable device. The computer software can be provided to the programmable device using any conventional carrier medium (carrier means). The carrier medium includes a transient carrier medium such as an electrical, optical, microwave, acoustic or radio frequency signal carrying the computer code. An example of such a transient medium is a Transmission Control Protocol/Internet Protocol (TCP/IP) signal carrying computer code over an IP network, such as the Internet. The carrier medium may also include a storage medium for storing processor readable code such as a floppy disk, a hard disk, a compact disc read-only memory (CD-ROM), a magnetic tape device, or a solid state memory device.

The functionality of the elements disclosed herein may be implemented using circuitry or processing circuitry which includes general purpose processors, special purpose processors, integrated circuits, application specific integrated circuits (ASICs), digital signal processors (DSPs), field programmable gate arrays (FPGAs), conventional circuitry and/or combinations thereof which are configured or programmed to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

This patent application is based on and claims priority to Japanese Patent Application Nos. 2022-192273, filed on Nov. 30, 2022, and 2023-173627, filed on Oct. 5, 2023, in the Japan Patent Office, the entire disclosure of each of which is hereby incorporated by reference herein.

### REFERENCE SIGNS LIST

1 Arousal state control system
10, 10A. 10B, 10C, 1000, 2000 Wearable device
12, 12a, 12b. 12c, 1004, 2003 Stimulator (Stimulation unit)
101 Curved portion
110, 1001, 2001 Body
115, 1005, 2004 Sensor
116 Cable
117 Communication device
120 Microprocessor
121, 122 First electrode
123, 124 Second electrode
125, 126 Third electrode
130 Arm
200 Information processing device
201 Processor
202 Main memory
203 Auxiliary memory
204 Input/output interface
205 Communication interface
210 Stimulation control unit
211 Biological information acquiring unit
212 Target level setting unit
213 Data analyzing unit
214 Stimulus determining unit
215 Evaluation acquiring unit
1002, 2002 Attaching unit
1003 Operation unit

1. An arousal state control system, comprising:
a sensor to acquire biological information of a user;
stimulation processing circuitry configured to determine, based on the biological information, one or more types of stimuli to control a state of arousal of the user; and
a stimulator to apply the one or more types of stimuli determined by the stimulation processing circuitry to the user.

2. The arousal state control system according to claim 1,
wherein the stimulation processing circuitry includes arousal state acquiring circuitry unit configured to analyze the biological information to acquire the state of arousal of the user, and
wherein the stimulation processing circuitry is configured to:
select a stimulus that increases the state of arousal of the user in a case where the state of arousal of the user is lower than a target state of arousal; and
select a stimulus that decreases the state of arousal of the user in a case where the state of arousal of the user is higher than the target state of arousal.

3. The arousal state control system according to claim 1,
wherein the stimulation processing circuitry is configured to determine, based on the biological information, a first stimulus that increases the state of arousal of the user and a second stimulus that decreases the state of arousal of the user, and
wherein the stimulator is to simultaneously or alternately apply the first stimulus and the second stimulus, as the one or more types of stimuli.

4. The arousal state control system according to claim 1,
wherein the stimulation processing circuitry is configured to acquire a setting of a target state of arousal according to a work type of the user; and
determine the one or more types of stimuli based on a change in the biological information with respect to the setting of the target state of arousal.

5. The arousal state control system according to claim 1,
wherein the stimulation processing circuitry is configured to:
acquire a setting of a target state of arousal according to a work type of the user; and
correct at least one of a stimulus type, a duration of stimulation, and a stimulus strength, of at least one of the one or more types of stimuli based on a change in the biological information with respect to the setting of the target state of arousal.

6. The arousal state control system according to claim 1,
wherein the stimulation processing circuitry is configured to determine two or more types of thermal stimuli or electrical stimuli based on the biological information, and
wherein the stimulator is to simultaneously or alternately apply the two or more types of thermal stimuli or electrical stimuli, as the one or more types of stimuli.

7. The arousal state control system according to claim 1,
wherein the stimulator is to apply the one or more types of stimuli to a neck of the user.

8. A wearable device comprising:
a body;
a first stimulator at a first portion of the body to apply a first stimulus; and
a second stimulator at a second portion of the body to apply a second stimulus different from the first stimulus,
one or both of the first stimulator stimulation unit and the second stimulator being driven based on biological information of a user wearing the body.

9. The wearable device according to claim 8, further comprising:
a sensor to acquire the biological information; and
communication circuitry configured to:
transmit the biological information to an information processing device; and
receive, from the information processing device, a control signal that drives one or both of the first stimulator and the second stimulator.

10. The wearable device according to claim 8, further comprising processing circuitry configured to:
acquire the biological information from a sensor and analyze the biological information; and
drive one or both of the first stimulator and the second stimulator stimulation unit based on a result of analysis of the biological information.

11. The wearable device according to claim 8, further comprising:
processing circuitry configured to analyze the biological information; and
a biological sensor communicably connected to the processing circuitry,
wherein the processing circuitry is configured to:
analyze the biological information based on an output of the biological sensor; and
drive one or both of the first stimulator and the second stimulator based on a result of analysis of the biological information.

12. An arousal state control method, comprising:
acquiring biological information of a user;
analyzing the biological information;
determining one or more types of stimuli based on the analyzed biological information, to control a state of arousal of the user; and
applying the determined one or more types of stimuli to the user.

13. The arousal state control method according to claim 12,
wherein the analyzing includes analyzing the biological information to acquire the state of arousal of the user, and
wherein the determining includes:
selecting a stimulus that increases the state of arousal in a case where the state of arousal is lower than a target state of arousal; and
selecting a stimulus that decreases the state of arousal in a case where the state of arousal is higher than the target state of arousal.

14. The arousal state control method according to claim 12,
wherein the applying includes simultaneously or alternately applying, to the user with a stimulator, a first stimulus that increases the state of arousal of the user and a second stimulus that decreases the state of arousal of the user, as the determined one or more types of stimuli.
